(19) United States
(12) Patent Application Publication
(10) Pub. No.: US 2009/0269297 A1
(43) Pub. Date: Oct. 29, 2009

(54) MULTIPLE COMPONENT COMPOUND AND METHOD FOR NEUTRALIZING OFFENSIVE ODORS

(76) Inventor: Donald Conover, SR., (US)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/109,715

(22) Filed: Apr. 25, 2008

(51) Int. Cl. A61L 9/012

(52) U.S. Cl. 424/76.21; 512/4

(57) ABSTRACT

A multiple component compound containing a molecular encapsulator and an aromatic complex is able to neutralize a wide variety of offensive odors, called malodors. The molecular encapsulator encapsulates molecules of malodors, including those derived from substances with sulfur groups or ammonia groups. The aromatic complex, via charge exchanges, disrupts the bonds of molecules of other malodors, including those derived from substances with fatty acids. Also, the compound may include a fragrance component, which would mask any remaining odor not addressed by the other components and would provide an overall fragrance as is desired.

### BACKGROUND OF THE INVENTION

1. Field of the Invention

The present disclosure relates generally to a chemical compound and method for neutralizing offensive odors, often referred to as malodors. There is a desire to neutralize many types of malodors by using a single compound.

2. Description of the Related Art

For thousands of years people have been using various methods of getting rid of offensive odors. Offensive odors range from body odors, to cigar smoke, to odors coming from kitchens or bathrooms, to odors found in garages and odors associated with garbage removal. Historically, and up until the present, the preferred methods to escape these noisome odors have been to mask the odor by providing large quantities of a pleasing aroma, or to release an odor that blends with the offensive odor to result in a different, more desirable aroma. Although these methods are conceptually simple, they often are less than completely effective, meaning that the offensive odor is still detectible, if only at a diminished level. Also, particularly if large quantities of a masking fragrance must be used, the masking fragrance itself may become offensive if it is present in significant strength. In some cases, adding a significant amount of a masking odor to an already-strong offensive odor results in a total odor level that itself is unpleasant. Because of these downsides, a lingering need exists for providing compositions and methods for addressing offensive odors that do not simply mask the odor, but rather neutralize the odors themselves.

Modem science allows classification of offensive odors. Many particularly offensive odors are caused by compounds that have a tendency either to donate or to accept protons; these compounds are known as “malodors.” For purposes of this patent, “malodor” is defined as an odor-causing compound composed at least partially of molecules that have a tendency to donate or accept protons, and whose odor is offensive to the average person. Examples of malodors include lower carboxylic acids, thiols, thiophenols, phenols, lower amines, phosphines, and arsines. Specific malodors include acetaldehyde, allyl mercaptan, ammonia, amyl mercaptan, benzyl mercaptan, butylamine, cadaverine, chlorine, chlorophenol, crotyl mercaptan, dibutylamine, diisopropylamine, dimethylamine, dimethyl sulfide, diphenyl sulfide, ethylamine, ethyl mercaptan, hydrogen sulfide, indole, isovaleric acid, methylamine, methyl mercaptan, ozone, propyl mercaptan, putrescine, pyridine, skatole, sulfur dioxide, tert-butyl mercaptan, thiocresol, thiophenol, triethylamine, urea, and valeric acid.

### SUMMARY OF THE INVENTION

The present invention relates generally to a malodor-neutralizing compound comprising a molecular encapsulator at least one aromatic complex combined with the molecular encapsulator. In a preferred embodiment, the molecular encapsulator is Ordenone, the trade name of a molecular encapsulator marketed by Belle Aire Fragrances, Inc. In another embodiment, the malodor-neutralizing compound contains a fragrance component, which masks any scent that might remain and provides an overall pleasant aroma to the malodor-neutralizing compound. By combining these three components, a single compound can be used to neutralize malodors derived from a wide variety of malodors, including malodors derived from sulfur groups (such as sulfides and thiazols), malodors derived from fatty acid groups (such as isovaleric acid), other malodors listed above, and malodors derived from unrelated functional groups.

The present invention also relates generally to a method of neutralizing malodors, by encapsulating the malodor, or by disrupting the bonds of the malodor. It is also envisioned that the method might include using a fragrance component to mask any remaining scent and to provide a desired overall aroma.

### DETAILED DESCRIPTION

Embodiments and applications of the present invention are limited only by the ways in which malodors present themselves. It is envisioned that the malodor-neutralizing compound can be added to a variety of substances in order to provide or enhance odor-neutralizing capabilities. Examples of products that can contain the malodor-neutralizing compound include room deodorants, whether aerosol sprays, wick-type liquids, wax-based solids, powders, or gels; detergents, including laundry detergents and fabric softeners; cleansers and disinfectants, such as toilet bowl cleaners and kitchen cleaners; bathroom accessories, such as bathroom tissues, sanitary napkins, towelettes, disposable diapers, and diaper pails; personal care products, such as antiperspirants and underarm deodorants, body deodorants, hair care products, foot care products (including powders, liquids, or colognes), aftershaves, and soaps; odor control products for use in manufacturing processes, including textile finishing, printing, and effluent control (as in stock yards and meat processing, sewage treatment, and garbage disposal); agricultural and pet care products, such as hen house effluents and animal litter; and large-scale closed air systems, as in gymnasiums and other large enclosures.

In an embodiment of the present invention, a multiple component compound is provided that neutralizes a variety of different offensive odors, particularly those associated with malodors. The malodor-neutralizing compound has at least two, and preferably three components. The first component is a molecular encapsulator, such as Ordenone, that operates on contact. The second is an aromatic complex that operates in the vapor phase. The third is a fragrance component, which acts on a cosmetic level to provide any desirable scent the user might want. The third component-the fragrance component-is optional; it is envisioned that the fragrance component will be present in some embodiments of the invention and absent from others. The compound can be made by combining the molecular encapsulator with the aromatic complex, as well as the fragrance component, if desired. In the context of this patent, “combining” means partially or completely joining as with chemical bonds, as well as by simply mixing the components mechanically without the components being chemically bound. Also, for purposes of this patent, the term “compound” includes components combined with our without chemical bonding; thus, components of a “compound” might be partially or completely bonded chemically, or they might be mechanically combined only.

The marked versatility of the malodor-neutralizing compound allows the malodor-neutralizing compound to be included in any number of products in any number of areas of application. For example, the malodor-neutralizing compound can be used in trash removal products (the nature of which would invariably involve a very diverse number of types of odor-causing smells), deodorants and personal care products that address pronounced odors stemming from fatty acids, and use of the compound alone. Because of the notable versatility of the malodor-neutralizing compound, the applications of this compound are varied and ubiquitous. In addition to incorporating the malodor-neutralizing compound into various products as listed above, the compound can be used to address odors in a variety of applications. Examples of situations in which the use of the malodor-neutralizing compound would be advantageous include hard surface cleaning (as with bathroom cleaners, tile cleaners, mop treatments, fiberglass cleaners, vinyl cleaners, and the like); deodorizing (as with air fresheners, absorbents, deodorant blocks, drain and trap treatments, garbage receptacles, bed pan odor treatments, incontinent care products, pet odor treatments, vomit cleaners, urinal and commode treatments, smoke and fire restoration products, and the like); situations requiring aerosol application (as with air fresheners, disinfectants, deodorizers, textile deodorizers, and the like); textile cleaning (as with fabric softeners, detergents, spot cleaners, upholstery shampoos, specialty products for nursing homes and hospitals, carpet cleaning products, locker room/health club products, and the like); cleansing (as with dishwashing soaps, hand cleaners, lotion soaps, bar soaps and concentrated liquids, and the like); and industrial processes (as with rendering, food processing, portable toilets/holding tanks for vehicles, paper processing, water and waste treatment, and the like).

According to one embodiment of the present invention, the malodor-neutralizing compound includes three components that enable it to neutralize effectively a variety of malodors. Because the malodor-neutralizing compound can be made by mechanically mixing the various components, it is envisioned that the components might not necessarily have any particular spatial arrangement. The components may exist in a non-linear, unsystematic arrangement and still remain effective, and the components need not be chemically or physically bonded.

According to one embodiment of the present invention, the first component, the molecular encapsulator, is Ordenone. Ordenone is a highly-concentrated molecular encapsulator owned and distributed by Belle Aire Fragrances, Inc., Mundelein, Ill. Because of its structure, Ordenone has the ability to encapsulate and neutralize many malodors-for example, malodors derived from sulfur or ammonia groups (e.g., sulfides, thiazoles, amines)-although Ordenone itself has no odor. This makes Ordenone useful for eliminating “fishy” or “putrid” smells, such as those that would be caused by malodors derived from sulfur or ammonia groups. Moreover, Ordenone, as a liquid, operates on contact, and is effective at eliminating odors emanating from the surfaces of solids, i.e., the area onto which the Ordenone is applied.

According to another embodiment of the present invention, the malodor-neutralizing compound is combined with the aromatic complex. A variety of aromatic complexes can be used to effectively neutralize odors in the vapor phase. Table 1 lists a variety of aromatic complexes that can be used in the malodor-neutralizing compound.

TABLE 1
Chemicals for Use as Aromatic Complexes.

| | | Molecular | |
|---|---|---|---|
| Common Name | IUPAC Name | Formula | Structure |
| Benzaldehyde | Benzaldehyde | C₆H₅CHO | 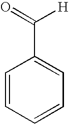 |
| Bourgeonal | 3-(4-tert-butylphenyl) propanal | C₁₃H₁₈O | 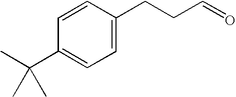 |
| Cinnamaldehyde | (E)-3-phenyl-2-propenal | C₉H₈O | 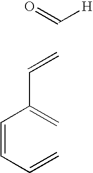 |
| Hexyl Cinnamaldehyde | (2E)-2- benzylideneoctanal | C₁₅H₂₀O | 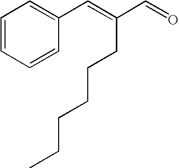 |
| Citronellal | 3,7-dimethyloct-6-en-1- al | C₁₀H₁₈O | 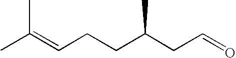 |
| Hydroxy Citronella | 7-hydroxy-3,7- dimethyloctanal | C₁₀H₂₀O₂ | 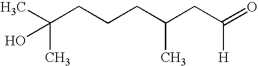 |
| Citral | 3,7-dimethyl-2,6- octadienal | C₁₀H₁₆O | 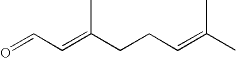 |
| Cuminaldehyde | 4-(1-methylethyl) benzaldehyde | C₁₀H₁₂O | 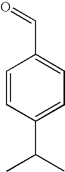 |
| Decanal | Decanal | C₉H₁₉CHO | 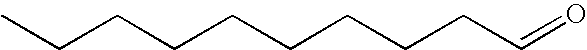 |
| Eugenol | 2-methoxy-4-prop-2- enyl-phenol | C₁₀H₁₂O₂ | 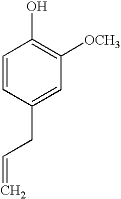 |
| Geraniol | 3,7-dimethyl-2,6- octadien-1-ol | C₁₀H₁₈O | 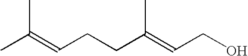 |
| Heptanal | Heptanal | C₇H₁₄O | 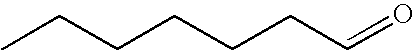 |
| Cis-3-hexen-1-ol | (Z)-hex-3-en-1-ol | C₆H₁₂O | 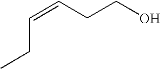 |
| Hexanal | Hexanal | C₆H₁₂O | 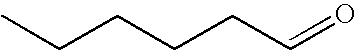 |
| β-Ionone | (3E)-4-(2,6,6- trimethylcyclohex-1-en- 1-yl) but-3-en-2-one | C₁₃H₂₀O | 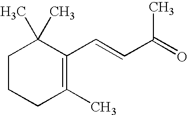 |
| γ-Ionone | (3E)-4-(2,2-dimethyl-6- methylenecyclohexyl) but-3-en-2-one | C₁₃H₂₀O | 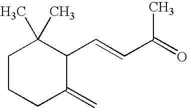 |
| Nonanaldehyde | Nonanal | C₉H₁₈O | 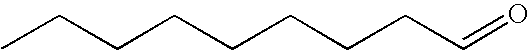 |
| Octanaldehyde | Octanal | C₈H₁₆O | 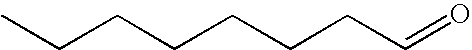 |
| Valeraldehyde | Pentanal | C₅H₁₀O | 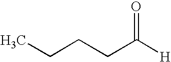 |
| Perillaldehyde | (S)-4-(1- methylethenyl)-1- cyclohexene- 1-carboxaldehyde | C₁₀H₁₅O | 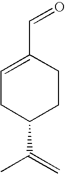 |
| Piperonal | 1,3-benzodioxole-5- carbaldehyde | C₈H₆O₃ | 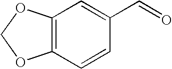 |
| Vanillin | 4-hydroxy-3- methoxybenzaldehyde | C₈H₈O₃ | 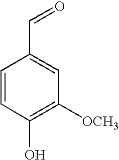 |

Although one embodiment of the present invention involves the addition of a single aromatic complex to the malodor-neutralizing compound, it is also envisioned that more than one aromatic complex may be used. In a preferred embodiment, the aromatic complex or complexes are selected from the given in Table 2, which gives aromatic complexes in addition to those listed in Table 1. Table 2 lists respective concentration ranges, in percentages by weight, at which various aromatic complexes are most effective at reducing fatty acid based odors, as well as a range, in percentage by weight, of concentrations in which Ordenone is most effective.

TABLE 2
Effective Amounts of Components

| | | Molecular | | Effective |
|---|---|---|---|---|
| Common Name | IUPAC Name | Formula | Structure | Range (% wt) |
| Ordenone | N/A | N/A | N/A | 0.04%-11.75% |
| Para tert-amyl cyclohexanone | 4-(tert-pentyl)- cyclohexanone | C₁₁H₂₀O | 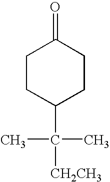 | 0.06%-1.00% |
| Ortho tert-butyl cyclohexanol | 2-tert- butylcyclohexanol | C₁₀H₂₀O | 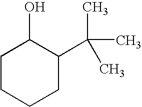 | 0.06%-1.00% |
| 3-Cyclohexene-1-carboxaldehyde 4-(4- hydroxy-4-methylpentyl); Lyral | 4-(4-hydroxy-4- methylpentyl)-3- cyclohexene-1- carboxaldehyde | C₁₃H₂₂O₂ | 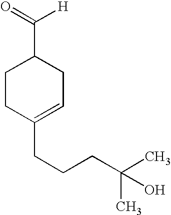 | 0.07%-0.35% |
| α-Methyl-4-(1-methylethyl) benzenepropanal | 2-methyl-3-(p- isopropylphenyl) propionaldehyde | C₁₃H₁₈O | 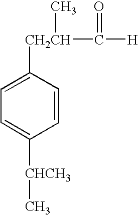 | 0.06%-1.00% |
| α-Ionone | 4-(2,6,6- trimethyl-2- cyclohexenyl)-3- buten-2-one | C₁₃H₂₀O | 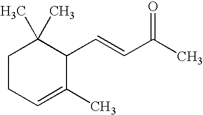 | 0.14%-0.80% |
| Para tert-butyl-alpha- methyldihydrocinnamic aldehyde | 2-(4-tert- butylbenzyl) propionaldehyde | C₁₄H₂₀O | 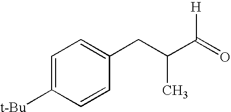 | 0.07%-0.35% |
| 4-Tert-butyl cyclohexanol | 4-tert-butyl cyclohexanol | C₁₀H₂₀O | 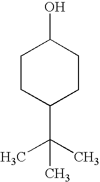 | 0.14%-0.80% |

The combination of the aromatic complex or complexes with Ordenone has several benefits. These benefits are more than simply the addition of a particular scent (e.g., fruity or almond). First, although the liquid Ordenone is very effective in operating on contact, the aromatic complex allows the malodor-neutralizing compound to operate in a vapor phase, thus increasing the malodor-neutralizing compound's effectiveness. This benefit is noteworthy; although Ordenone alone is very effective at eliminating fishy or putrid odors on contact, it is less effective at eliminating odors caused by fatty acids, such as isovaleric acid. The aromatic complex operates through a process of chemical/electron charge exchange, and this works with Ordenone to eliminate odors from lower fatty acids, such as isovaleric acid. Malodors have a tendency to either donate or accept protons due to the presence of polar groups on these molecules. On these smaller molecules, a polar group will play an important part of any interaction that can occur with other compositions. Because of this, any exchange of protons with this polar group on the malodorous molecule will temper the malodor's tendency to cause a foul odor.

The functional group on the aromatic complex also contains polar groups that, when they interact with the malodors, accept or donate protons, thus causing bond disruptions in the fatty acid. In particular, carbonyl groups as would be present in aldehydes are effective to cause this bond disruption effect. Also aldehydes often are associated with pleasant smells, which may make particular aldehydes desirable. Even aldehydes that have offensive odors—such as butyraldehyde—may be effective at causing these bond disruptions; even though the use of foul smelling aldehydes would not be appropriate in some applications of the present invention, in other applications the scent associated with the aldehyde used might be irrelevant.

A second beneficial effect of combining the molecular encapsulator, Ordenone, with the aromatic complex is that the aromatic complex enhances the effectiveness of Ordenone on certain malodors. Although the bond disruptions caused by the aromatic complex do not change the identity of the malodorous molecules themselves, the bond disruptions caused by the aromatic complex render fatty acids (or other malodor) more susceptible to encapsulation by the molecular encapsulator, such as Ordenone. In other words, the addition of the aromatic complex does more than provide a masking scent, but rather it works with Ordenone to eliminate not only the malodors that Ordenone eliminates on its own, but a wide variety of malodors that Ordenone ordinarily would not eliminate (or would eliminate only partially). Thus, the advantages of this combination are more than simply adding two individually beneficial components.

The compound formed by the combination of the molecular encapsulator and the aromatic complex is not a masking agent, nor does it simply dull a person's sense of smell. However, the malodor-neutralizing compound can be combined with a fragrance in order to produce whatever scent is desired by the user. One embodiment of the malodor-neutralizing compound includes a fragrance component. The fragrance component may be one or more of any number of fragrances, such as a commercially available perfume, that partially or completely covers any unpleasant odor, as well as any malodor that might not be neutralized by the molecular encapsulator or the aromatic complex. Although the molecular encapsulator and aromatic complex typically work in concert to eliminate a noisome malodor, the addition of the fragrance component nonetheless provides a pleasant smell, such as lilac or sandalwood, or a synthetic scent, which might make the malodor-neutralizing compound more desirable depending on the context in which it is used.

The malodor-neutralizing compound is advantageous in that the components work in conjunction-not independently of each other-in order to effectively neutralize a wide variety of malodors by the use of a single compound. Thus, if a user encounters an offensive odor, the user need only use a single compound to address this odor, without concern as to exactly what types of chemicals comprise the odor. The user can be assured that, by using the malodor-neutralizing compound, all of the ammonia and sulfur based malodors will be neutralized by the molecular encapsulator on contact; all of the fatty acid based malodors will be neutralized by the aromatic complex working in conjunction with the molecular encapsulator in the vapor phase; and any desirable aroma will be provided by the fragrance component. Thus, by working together, the various components in the malodor-neutralizing compound each address different categories of malodors, such that the resulting compound effectively neutralizes almost any malodor that a user is likely to encounter, without regard as to the source or chemistry of the odor.

Because of the versatility offered by the combination of the various components, the compound has many practical uses. It is envisioned that the malodor-neutralizing compound can be added as an ingredient into another substance, such as personal care products like foot and underarm deodorants, pet shampoos, surface cleaners or deodorizers, room deodorizers, garbage receptacles and trash bags, and automobile fresheners. Although commonly envisioned as a liquid ingredient in products such as those listed above, it is also envisioned that the malodor-neutralizing compound can be used as a stand-alone product. In this form, the compound may enable such uses as industrial applications and use at waste landfills. Moreover, although the malodor-neutralizing compound is commonly envisioned as a liquid, it may be used in other forms as well, such as a gas or in an aerosol application, or in a granulated solid form. It may also be included in a microencapsulation application, such as an adhesive strip that selectively releases the malodor-neutralizing compound.

The present invention has been described in specific detail and with particular reference to its preferred embodiments; however, it will be apparent to those having skill in the art that modifications and changes can be made thereto without departing from the spirit and scope of the invention.

I claim:

1. A malodor-neutralizing compound comprising:
an molecular encapsulator that encapsulates the molecules of at least some malodors; and
at least one aromatic complex combined with the molecular encapsulator to form a single compound.

2. The malodor-neutralizing compound of claim 1, further comprising a fragrance component, wherein the molecular encapsulator and the aromatic complex or complexes are combined to form a single compound.

3. The malodor-neutralizing compound of claim 1, wherein the aromatic complex or complexes are selected from a group consisting of:
Benzaldehyde;
Bourgeonal;
Cinnamaldehyde;
Hexyl Cinnamaldehyde;
Citronellal;
Hydroxy Citronella;
Citral;
Cuminaldehyde;
Decanal;
Eugenol;
Geraniol;
Heptanal;
Cis-3-Hexen-1-ol;
Hexanal;
α-Ionone;
β-Ionone;
γ-Ionone;
Lyral;
Nonanaldehyde;
Octanaldehyde;
Valeraldehyde;
Perillaldehyde;
Piperanal;
Vanillin;
para tert-amyl cyclohexanone;
ortho tert-butyl cyclohexanol;
3-cyclohexene-1-carboxaldehyde 4-(4-hydroxy-4-methylpentyl);
alpha-methyl-4-(1-methylethyl) benzenepropanal;
para tert-butyl-alpha-methyldihydrocinnamic aldehyde;
4-tert-butyl cyclohexanol.

4. The malodor-neutralizing compound of claim 2, wherein the aromatic complex or complexes are selected from a group consisting of:
Benzaldehyde;
Bourgeonal;
Cinnamaldehyde;
Hexyl Cinnamaldehyde;
Citronellal;
Hydroxy Citronella;
Citral;
Cuminaldehyde;
Decanal;
Eugenol;
Geraniol;
Heptanal;
Cis-3-Hexen-1-ol;
Hexanal;
α-Ionone;
β-Ionone;
γ-Ionone;
Lyral;
Nonanaldehyde;
Octanaldehyde;
Valeraldehyde;
Perillaldehyde;
Piperanal;
Vanillin;
para tert-amyl cyclohexanone;
ortho tert-butyl cyclohexanol;
3-cyclohexene-1-carboxaldehyde 4-(4-hydroxy-4-methylpentyl);
alpha-methyl-4-(1-methylethyl)benzenepropanal;
para tert-butyl-alpha-methyldihydrocinnamic aldehyde;
4-tert-butyl cyclohexanol.

5. The malodor-neutralizing compound of claim 3, wherein the aromatic complex or complexes are selected from a group consisting of para tert-amyl cyclohexanone, ortho tert-butyl cyclohexanol, and alpha-methyl-4-(1-methylethyl)benzenepropanal, and wherein the aromatic complex or complexes are provided in a range of about 0.06% by weight to about 1.00% by weight.

6. The malodor-neutralizing compound of claim 3, wherein the aromatic complex or complexes are selected from a group consisting of 3-cyclohexene-1-carboxaldehyde 4-(4-hydroxy-4-methylpentyl) and para tert-butyl-alpha-methyldihydrocinnamic aldehyde, and wherein the aromatic complex or complexes are provided in a range of about 0.07% by weight to about 0.35% by weight.

7. The malodor-neutralizing compound of claim 3, wherein the aromatic complex or complexes are selected from a group consisting of 4-(2,6,6-trimethyl-2-cyclohexenyl)-3-buten-2-one and 4-tert-butyl cyclohexanol, and wherein the aromatic complex or complexes are provided in a range of about 0.14% by weight to about 0.80% by weight.

8. The malodor-neutralizing compound of claim 4, wherein the aromatic complex or complexes are selected from a group consisting of para tert-amyl cyclohexanone, ortho tert-butyl cyclohexanol, and alpha-methyl-4-(1-methylethyl) benzenepropanal, and wherein the aromatic complex or complexes are provided in a range of about 0.06% by weight to about 1.00% by weight.

9. The malodor-neutralizing compound of claim 4, wherein the aromatic complex or complexes are selected from a group consisting of 3-cyclohexene-1-carboxaldehyde 4-(4-hydroxy-4-methylpentyl) and para tert-butyl-alpha-methyldihydrocinnamic aldehyde, and wherein the aromatic complex or complexes are provided in a range of about 0.07% by weight to about 0.35% by weight.

10. The malodor-neutralizing compound of claim 4, wherein the aromatic complex or complexes are selected from a group consisting of 4-(2,6,6-trimethyl-2-cyclohexenyl)-3-buten-2-one and 4-tert-butyl cyclohexanol, and wherein the aromatic complex or complexes are provided in a range of about 0.14% by weight to about 0.80% by weight.

11. A method of neutralizing malodors, comprising the steps of
if the malodor contains molecules having sulfur or ammonia groups, encapsulating the molecules having sulfur or ammonia groups; and
if the malodor contains molecules having fatty acid groups, disrupting the bonds of the fatty acid by exchanging charges with the molecules having a fatty acid group and encapsulating the molecules having sulfur or ammonia groups.

12. The method of neutralizing malodors of claim 11, further comprising the step of
if the malodor contains molecules having sulfur or ammonia groups or fatty acid groups or neither sulfur or ammonia groups nor fatty acid groups, masking the malodor and any other odor caused by the performance of the previous steps.

13. The method of neutralizing malodors of claim 11, further comprising the step of using a fragrance component to provide an overall desirable fragrance.

14. A malodor-neutralizing compound comprising:
Ordenone, provided in a range of about 0.04% by weight to about 11.75% by weight;
at least one aromatic complex combined with the Ordenone to form a single compound, wherein the aromatic complex or complexes are selected from a list consisting of
Benzaldehyde;
Bourgeonal;
Cinnamaldehyde;
Hexyl Cinnamaldehyde;
Citronellal;
Hydroxy Citronella;
Citral;
Cuminaldehyde;
Decanal;
Eugenol;
Geraniol;
Heptanal;
Cis-3-Hexen-1-ol;
Hexanal;
α-Ionone;
β-Ionone;
γ-Ionone;
Lyral;
Nonanaldehyde;
Octanaldehyde;
Valeraldehyde;
Perillaldehyde;
Piperanal;
Vanillin;
para tert-amyl cyclohexanone;
ortho tert-butyl cyclohexanol;
3-cyclohexene-1-carboxaldehyde 4-(4-hydroxy-4-methylpentyl);
alpha-methyl-4-(1-methylethyl) benzenepropanal;
para tert-butyl-alpha-methyldihydrocinnamic aldehyde;
4-tert-butyl cyclohexanol; and
a fragrance component combined with the Ordenone and aromatic complex or complexes;
wherein the Ordenone, aromatic complex or complexes, and fragrance component are combined to form a single compound.

15. A malodor-neutralizing compound comprising:
a molecular encapsulator; and
an aldehyde combined with the molecular encapsulator to form a single compound.

16. The malodor-neutralizing compound of claim 15, further comprising a fragrance component, wherein the molecular encapsulator, the aldehyde, and the fragrance component are combined to form a single compound.

17. The malodor-neutralizing compound of claim 15, wherein the molecular encapsulator is Ordenone.

18. The malodor-neutralizing compound of claim 15, wherein the aldehyde itself has a desirable fragrance.

19. A malodor-neutralizing compound comprising:
A molecular encapsulator; and
A chemical whose functional group includes a carbonyl group.

20. The malodor-neutralizing compound of claim 19, wherein the chemical whose functional group includes a carbonyl group itself has a desirable fragrance.

21. The malodor-neutralizing compound of claim 19, further comprising a fragrance component, wherein the molecular encapsulator, the chemical whose functional group includes a carbonyl group, and the fragrance component are combined to form a single compound.
